(19) United States
(12) Patent Application Publication
(10) Pub. No.: US 2025/0073160 A1
(43) Pub. Date: Mar. 6, 2025

(54) COMPOSITION FOR IMPROVING SKIN COMPATIBILITY

(71) Applicant: Yamada Bee Company, Inc., Tomata-gun (JP)

(72) Inventors: Masayuki YAGI, Tomata-gun (JP); Yuka MAEDA, Tomata-gun (JP)

(21) Appl. No.: 18/824,373

(22) Filed: Sep. 4, 2024

(30) Foreign Application Priority Data
Sep. 6, 2023 (JP) 2023-144569

(51) Int. Cl. A61K 8/98; A61Q 19/00

(52) U.S. Cl. CPC A61K 8/988; A61Q 19/00; A61K 2800/10

(57) ABSTRACT

The present invention provides a composition for improving skin compatibility, comprising: honey, in which a contact angle of water on the skin to which the composition has been applied is 30 degrees or less. The present invention also provides a method for improving skin compatibility, comprising applying the composition to the skin.

### CROSS-REFERENCE TO RELATED APPLICATION

The present application claims priority under 35 U.S.C. § 119 to Japanese Application No. 2023-144569 filed on Sep. 6, 2023, the entire contents of which being incorporated herein by reference.

### TECHNICAL FIELD

The present invention relates to a composition for improving skin compatibility.

### BACKGROUND

The stratum corneum of the skin acts as a barrier against water evaporation from the body and the entry of viruses, substances, and the like from outside, making it difficult to deliver a sufficient amount of compounds, such as pharmaceuticals or cosmetics, from outside the skin to inside the skin. On the other hand, there are also cosmetics on the market that are designed to promote permeability of ingredients in a formulation to be applied next into the skin (also called booster serums or introduction liquids (introduction serums)). Such cosmetics contain, for example, surfactants, polyols, and lactic acid.

For example, Japanese Patent No. 5022583, Patent Document 1 discloses, as a subsequent method to enhance the compatibility and permeability of skin care cosmetic materials, a skin care pretreatment method including: (1) a step of washing the skin with a skin cleanser and then rinsing off the cleanser with water; (2) a step of applying a skin rinse composition, which has a liquid-like oil phase at normal temperature and an aqueous phase and in which the oil phase and the aqueous phase are separated into two layers, the content of the oil phase is 3 to 30 mass %, and the oil phase contains 1 mass % or more of higher fatty acids in a liquid form at normal temperature, onto the skin in a shaken and dispersed state while the skin is still wet; and (3) a step of rinsing off the skin rinse composition with water.

In relation to compositions to be applied to the skin, for example, Japanese Patent No. 6888835, Patent Document 2 discloses a fermentation product obtained by fermenting honey and chamomile or a processed product thereof with Lactobacillus kunkeei, and states that the fermentation products can be used for skin barrier function improvement, skin moisturization, skin elasticity improvement, skin dullness improvement, skin wrinkle improvement, skin roughness improvement, skin aging prevention, skin texture improvement, and sensitive skin improvement. Furthermore, O'Farrell C, et al., “Formulation of an antibacterial topical cream containing bioengineered honey that generates reactive oxygen species” Biomater Adv. 2022 February; 133:112664. doi:10.1016/j. msec. 2022. 112664. Epub 2022 Jan. 20, Non-Patent Document 1 describes that a cream containing bioengineered medicinal honey that has been proven to sterilize multi-drug resistant bacterial strains by delivering active oxygen species shows a low contact angle on porcine skin while maintaining the sterilization action, and favorably spreads on the skin surface.

### SUMMARY

An object of the present invention is to provide a novel composition for improving skin compatibility.

The present inventors have conducted extensive studies to achieve the above-described object, and as a result, they have found that the contact angle of water on an artificial skin model to which honey has been applied is 30 degrees or less after applying honey to the artificial skin model, thus leading to realization of the present invention.

That is, the present invention relates to, for example, the following inventions.
[1]A composition for improving skin compatibility, comprising: honey, in which a contact angle of water on the skin to which the composition has been applied is 30 degrees or less.
[2] The composition according to [1], further comprising: royal jelly; butylene glycol; and pentylene glycol.
[3] The composition according to [1] or [2], further comprising: Lactobacillus fermented extract of honey.
[4] The composition according to [3], in which the Lactobacillus fermented extract of honey is obtained through fermentation of honey and chamomile flower with Lactobacillus kunkeei.
[5] The composition according to any one of [1] to [4], in which the honey is Manuka honey and/or acacia honey.
[6] The composition according to any one of [1] to [5], which comprises based on the total amount of the composition, 5% by mass or more and 60% by mass or less of honey, 0.001% by mass or more and 0.5% by mass or less of royal jelly, 0.1% by mass or more and 10% by mass or less of butylene glycol, 0.01% by mass or more and 2.5% by mass or less of pentylene glycol, and 25% by mass or more and 35% by mass or less of a Lactobacillus fermented extract of honey.
[7]A method for improving skin compatibility, comprising applying the composition according to any one of [1] to [6] to the skin.
[8]A method for improving skin compatibility by applying a composition to the skin, comprising blending honey into the composition, and in which a contact angle of water on the skin to which the composition has been applied is 30 degrees or less.
[9] The method according to [8], comprising blending royal jelly; butylene glycol; and pentylene glycol into the composition.
[10] The method according to [8] or [9], further comprising blending based on the total amount of the composition, 5% by mass or more and 60% by mass or less of honey, 0.001% by mass or more and 0.5% by mass or less of royal jelly, 0.1% by mass or more and 10% by mass or less of butylene glycol, 0.01% by mass or more and 2.5% by mass or less of pentylene glycol, and 25% by mass or more and 35% by mass or less of a Lactobacillus fermented extract of honey into the composition.

According to the present invention, it is possible to provide a novel composition for improving skin compatibility.

### DETAILED DESCRIPTION

Hereinafter, embodiment of the present invention will be described in detail. However, the present invention is not limited to the following embodiment.

### [Composition]

A composition for improving skin compatibility according to the present embodiment (hereinafter also referred to as ‘composition’) includes honey, in which the contact angle of water on the skin to which the composition has been applied is 30 degrees or less.

The composition according to the present embodiment has a water contact angle of 30 degrees or less on the skin to which the composition has been applied, and has an effect of improving skin compatibility. Here, the expression “improving skin compatibility” in the present specification means improving the compatibility of hydrophobic or hydrophilic liquids with the skin, and it can be evaluated by whether the contact angle of water on the skin when water is added dropwise onto the skin to which the above-described composition has been applied becomes smaller than the contact angle thereof on the skin to which the above-described composition has not been applied. If the above-described contact angle of water is decreased after applying the above-described composition, it can be determined that the skin compatibility has improved. In addition, the contact angle of water is generally used as an indicator for wettability evaluation. Therefore, the composition according to the present embodiment can also be rephrased as a composition for improving the wettability of the skin.

In the measurement of the contact angle of water, the skin to which the composition according to the present embodiment is to be applied may be natural skin or artificial skin. In addition, natural skin may be human or animal skin, and preferably human skin. In the case of human skin, it may be skin of the cheeks, forearm, and the like and is preferably hairless (hair removed) skin. In the case of artificial skin, for example, it may be one that mimics human skin, may be artificial skin induced to differentiate by iPS cells, or may be an artificial skin model made of urethane. In determining that the contact angle of water on the skin to which the composition has been applied is 30 degrees or less, the contact angle of water is determined under the conditions that the skin is forearm skin or an artificial skin model made of urethane, 0.1 g of the composition is uniformly applied within 5 cm×5 cm on the skin, 2 μL of water is added dropwise thereto after 1 hour, and the measurement of the contact angle is performed immediately after dropwise addition of water (pure water such as distilled water) (1,000 milliseconds later).

The measurement of the contact angle of water can be performed using, for example, an automatic contact angle meter DMo-602 (manufactured by Kyowa Interface Science Co., Ltd.). The contact angle of water can be measured through, for example, a liquid droplet method. The contact angle of water can be analyzed (calculated) through, for example, a θ/2 method and a tangent method.

The contact angle of water on human skin varies depending on the part, but is around 60 degrees to 102 degrees. In addition, the contact angle of water on the human forearm is generally around 90 degrees, and artificial skin also has a contact angle of water close to that of the human forearm. It is also preferable to use animal skin other than human skin which has a water contact angle of around 90 degrees. The composition according to the present embodiment preferably reduces the contact angle of water on the skin when water is added dropwise onto the skin to which the composition has been applied by 50 degrees or more, 55 degrees or more, 60 degrees or more, or 65 degrees or more, compared to the contact angle of water on the skin to which the above-described composition has not been applied.

The composition according to the present embodiment may have a water contact angle on the skin to which the composition has been applied of 30 degrees or less, for example, 28 degrees or less, 26 degrees or less, 24 degrees or less, 22 degrees or less, 20 degrees or less, 18 degrees or less, 16 degrees or less, 14 degrees or less, or 13 degrees or less, and 0 degrees or more, 1 degree or more, 3 degrees or more, or 5 degrees or more.

The composition according to the present embodiment is intended to be applied to the skin and can be applied to any part of the skin. “Apply” in the present specification may mean to coat or to spray.

Honey is a substance produced mainly from nectar collected by bees from, for example, plant nectar, sap, and secretions from insects that parasitize plants.

The types of honey contained in the composition according to the present embodiment are not particularly limited, and for example, honeydew honey, Ulmo honey, Manuka honey, acacia honey, linden honey, rewarewa honey, multifloral honey, clover honey, orange honey, Chinese milk vetch honey, rosemary honey, sunflower honey, rape blossom honey, coffee honey, cilantro (coriander) honey, Japanese horse chestnut honey, buckwheat honey, Haze honey, Soyogo honey, Kuroganemochi honey, and Karasu Sansho honey can be used. Honey is preferably Manuka honey and/or acacia honey.

Manuka honey is honey that can be collected from flowers of Manuka (Leptospermum scoparium), which belongs to the genus Leptospermum in the family Myrtaceae. Acacia honey is honey that can be collected from flowers of false acacia (Robinia pseudoacacia), which belongs to the genus Robinia in the family Fabaceae.

The types of bees used to collect honey is not particularly limited. Honey, for example, can be obtained as a beekeeping product in accordance with usual methods.

Honey is generally kept at around 35° C. by, for example, the body temperature of bees while it is stored in beehives. Honey collected from beehives is generally heated as necessary to lower viscosity for facilitating operations such as transportation and filling, and to melt crystals.

The collected honey may be left as it is, or it may undergo purification treatments such as removal of proteins, wax, debris, bee or hive residues, filtration, extraction with organic solvents, and fractionation, and treatments such as sterilization and concentration, before or after heating.

Honey is known to be a substance with moisturizing properties, but it is not known to be a substance that improves skin compatibility.

The content of honey in the composition according to the present embodiment based on the total amount of the composition may be, for example, 5 mass % or more, 10 mass % or more, 15 mass % or more, 20 mass % or more, 25 mass % or more, or 30 mass % or more, and may be, for example, 100 mass % or less, 90 mass % or less, 80 mass % or less, 70 mass % or less, or 60 mass % or less.

The composition according to the present embodiment may further contain royal jelly, butylene glycol, and pentylene glycol. Accordingly, the contact angle of water on the skin to which the composition according to the present embodiment has been applied becomes smaller, and the skin compatibility is further improved.

The royal jelly in the present invention includes raw royal jelly, dried royal jelly, dried royal jelly powder, enzyme-treated royal jelly, royal jelly extract, fermented royal jelly, and the like. In addition, the production areas of royal jelly may be any of the following: European countries, Oceanian countries, America, Brazil, Japan, China, and other Asian countries.

Dried royal jelly powder is made by drying and powdering raw royal jelly. As a drying method, any of the well-known methods used in general food processing can be used, for example, natural drying like air drying and sun drying, forced drying through heating with electricity or the like, and freeze drying. Freeze drying is preferable.

Enzyme-treated royal jelly is royal jelly that has been treated with a proteolytic enzyme (protease). Preferably, it is hypoallergenic enzyme-treated royal jelly in which allergic reactions caused by proteins contained in royal jelly are suppressed by protease treatment. Therefore, enzyme-treated royal jelly may contain not only protease degradation products of proteins contained in royal jelly, but also organic acids such as unsaturated fatty acids and saturated fatty acids described above, lipids, sugars, vitamins, and various minerals.

Royal jelly used in the production of enzyme-treated royal jelly is not particularly limited, and examples thereof include raw royal jelly, royal jelly powder obtained by drying and powdering raw royal jelly, or raw royal jelly extracted with water, hydrated ethanol, or the like.

The production of enzyme-treated royal jelly can be carried out by treating a raw material of royal jelly with enzymes having at least endopeptidase activity, enzymes having at least exopeptidase activity, and/or enzymes having both endopeptidase activity and exopeptidase activity.

Examples of proteolytic enzymes having at least endopeptidase activity include endopeptidases derived from animals (for example, trypsin and chymotrypsin), plants (for example, papain), and microorganisms (for example, lactic acid bacteria, yeast, mold, Bacillus subtilis, and Actinomycetes).

Examples of proteolytic enzymes having at least exopeptidase activity include carboxypeptidase, aminopeptidase, exopeptidases derived from microorganisms (for example, lactic acid bacteria, Aspergillus bacteria, and Rhizopus bacteria), and pancreatin and pepsin which also have endopeptidase activity.

Among these various enzymes, preferred examples of enzymes having both exopeptidase activity and endopeptidase activity include peptidase produced by Streptomyces griseus (trade name: ACTINASE AS), peptidase produced by Aspergillus oryzae (trade names: PROTEASE A, and FLAVOURZYME, and PROTEAX), and peptidase produced by Aspergillus melleus (trade name: PROTEASE P), and preferred examples of enzymes having exoprotease activity include peptidase produced by Aspergillus oryzae (trade name: UMAMIZYME G, PROMOD 192P, PROMOD 194P, and SUMIZYME FLAP), peptidase produced by Aspergillus sojae (trade name: STERNZYME B15024), peptidase produced by the genus Aspergillus (trade name: KOKULASE P), and peptidase produced by Rhizopus oryzae (trade name: PEPTIDASE R). Furthermore, preferred examples of enzymes having endopeptidase activity include peptidase produced by Bacillus subtilis (trade name: ORIENTASE 22BF and NUCLEICIN), peptidase produced by Bacillus licheniformis (trade name: ALCALASE), peptidase produced by Bacillus stearothermophilus (trade name: PROTEASE S), peptidase produced by Bacillus amyloliquefaciens (trade name: NEUTRASE), and peptidase produced by the genus Bacillus (trade name: PROTAMEX).

Enzymatic treatment to reduce allergenicity of royal jelly can be carried out, for example, according to the descriptions in Japanese Unexamined Patent Publication No. 2007-295919 and Japanese Unexamined Patent Publication No. 2007-295920.

Royal jelly extract is obtained by extracting royal jelly (including raw, dried, and pulverized forms) with water, hydrated ethanol, or the like.

Fermented royal jelly can be produced through a usual method using microorganisms such as yeast and lactic acid bacteria.

The content of royal jelly in the composition according to the present embodiment based on the total amount of the composition may be, for example, 0.001 mass % or more, 0.005 mass % or more, 0.01 mass % or more, 0.05 mass % or more, 0.1 mass % or more, or 0.2 mass % or more, and may be, for example, 10 mass % or less, 5 mass % or less, 1 mass % or less, or 0.5 mass % or less.

Butylene glycol is a compound also known as butanediol, and is a substance often used as a humectant. In the present specification, butylene glycol refers to 1,3-butylene glycol. The content of butylene glycol in the composition according to the present embodiment may be, for example, 0.1 mass % or more, 0.5 mass % or more, 1 mass % or more, 2 mass % or more, 3 mass % or more, 4 mass % or more, 5 mass % or more, 6 mass % or more, 7 mass % or more, or 8 mass % or more, and may be, for example, 30 mass % or less, 25 mass % or less, 20 mass % or less, 15 mass % or less, or 10 mass % or less.

Pentylene glycol is a compound also known as 1,2-pentanediol, and is a substance often used as a humectant. The content of pentylene glycol in the composition according to the present embodiment may be, for example, 0.01 mass % or more, 0.05 mass % or more, 0.1 mass % or more, 0.5 mass % or more, 1 mass % or more, or 2 mass % or more, and may be, for example, 10 mass % or less, 8 mass % or less, 6 mass % or less, 4 mass % or less, or 2.5 mass % or less.

The composition according to the present embodiment may further contain Lactobacillus fermented extract of honey. Accordingly, the contact angle of water on the skin to which the composition according to the present embodiment has been applied becomes smaller, and the skin compatibility is further improved.

Lactobacillus fermented extract of honey is obtained by fermenting honey using Lactobacillus, and preferably obtained by fermenting honey and chamomile flower using Lactobacillus.

Chamomile is a plant of the family Compositae (or Asteraceae), and its scientific name is Matricaria chamomilla L. or Matricaria recutita L.

Chamomile flower to be fermented may be one obtained by collecting chamomile flower or may be processed chamomile flower. Examples of processed chamomile flower include processed products obtained by subjecting chamomile flower to processing such as drying, pulverization, and extraction. These kinds of processing may be performed alone or in combination of two or more thereof.

Processed chamomile flower may be, for example, dried products, pulverized products, crushed products, powders, and extracts of chamomile flower. Drying of chamomile flower can be, for example, performed through sun drying, hot air drying, freeze drying, and spray drying, and the like. Pulverizing and crushing can be performed using, for example, a pulverizer or a crusher. Chamomile flower is preferably dried powder of chamomile flower. Dried powder of chamomile flower can be obtained, for example, through drying and pulverizing chamomile flower. Chamomile flower is preferably dried and pulverized immediately after collection. As for processed chamomile flower, commercially available products can be used.

Chamomile flower extract can be obtained by extracting chamomile flower through a method generally used for plant extraction. For example, chamomile flower, which is raw material extract, can be placed into a treatment tank filled with an extraction solvent, soluble components can be eluted while appropriately performing stirring as necessary, filtering can be performed, and then extraction residue can be removed to obtain an extract.

As for chamomile flower extract, an extract obtained by extracting chamomile flower may be used as it is, or an extract further subjected to processing, for example, dilution, concentration, drying, filtration, and purification may be used.

As for the extraction solvent, for example, an aqueous solvent or a hydrophilic organic solvent may be used. Examples of aqueous solvents include pure water, purified water, tap water, well water, mineral spring water, mineral water, hot spring water, spring water, fresh water, hot water, ion-exchanged water, physiological saline, phosphate buffer solution, and phosphate-buffered physiological saline. Examples of hydrophilic organic solvents include C1-5 lower alcohols such as methanol, ethanol, propyl alcohol, and isopropyl alcohol; lower aliphatic ketones such as acetone and methyl ethyl ketone; C2-5 polyhydric alcohols such as 1,3-butylene glycol, propylene glycol, and glycerol. The extraction solvent may be used alone or in combination of two or more thereof.

Fermentation of honey or fermentation of honey and chamomile flower, is carried out using Lactobacillus. Examples of Lactobacillus include bacteria belonging to the genus Lactobacillus, and more specifically, Lactobacillus kunkeei is preferable.

Lactobacillus kunkeei preferably exhibits an ability to assimilate glucose and fructose and more preferably exhibits an ability to assimilate glucose, fructose, sucrose, trehalose, and gluconate. Lactobacillus kunkeei may be Lactobacillus kunkeei BPS402. Lactobacillus kunkeei BPS402 was deposited on Oct. 3, 2011 in the Patent Microorganisms Depositary of the National Institute of Technology and Evaluation (Central 6, 1-1, Higashi 1-chome, Tsukuba-shi, Ibaraki-ken (305-8566) Japan) as the accession number FERM P-22177, and is available. In addition, the bacterial strain has been transferred to international depositary, and the accession number is FERM BP-11439.

Culture of Lactobacillus used for fermentation can be carried out according to a usual method. There are no particular restrictions on media as long as the bacteria can be cultured, and natural media, synthetic media, semi-synthetic media, and the like can be used. As for the media, those containing a nitrogen source and a carbon source can be used. Examples of nitrogen sources include meat extract, peptones, casein, yeast extract, gluten, soybean flour, soybean hydrolysate, and amino acids. Examples of carbon sources include glucose, lactose, fructose, inositol, sorbitol, water, starch, koji juice, bran, bagasse, and molasses. In addition, inorganic substances (for example, ammonium sulfate, potassium phosphate, magnesium chloride, table salt, calcium carbonate, iron, manganese, and molybdenum), various vitamins, and the like can be added.

The culture temperature may be, for example, 4° C. to 45° C., preferably 25° C. to 40° C., and more preferably 28° C. to 33° C. The culture time may be, for example, 8 to 72 hours. The culture may be carried out through aeration shaking or aeration stirring. The pH of media may be, for example, 4.0 to 9.0, preferably 6.0 to 8.0. Examples of culture methods include inoculating MRS medium with bacterial cells to carry out culture at 30° C. for 48 hours.

The bacterial suspension obtained by culturing Lactobacillus may be used for fermentation by directly inoculating a fermentation medium containing honey with the bacterial suspension or inoculating a fermentation medium containing honey and chamomile flower or may be diluted with a medium or the like for use.

Fermentation of honey or fermentation of honey and chamomile flower by Lactobacillus can be carried out, for example, at 4° C. to 45° C., and is preferably carried out at 25° C. to 40° C. The fermentation time may be, for example, 12 to 60 hours, preferably 24 to 48 hours. The pH of the honey content at the start of fermentation may be, for example, 3 to 8, preferably 4 to 6. The fermentation may be carried out through aeration shaking or aeration stirring.

Fermentation of honey or fermentation of honey and chamomile flower can be carried out by culturing Lactobacillus in a fermentation medium containing honey or in a fermentation medium containing honey and chamomile flower. A fermentation medium containing honey or a fermentation medium containing honey and chamomile flower may be in any form that allows Lactobacillus to ferment, and may be, for example, in a form such as a liquid, a paste, or a gel. Fermentation media may be obtained by, for example, dissolving or suspending honey or honey and chamomile flower in a solvent such as water.

The concentration of honey in a fermentation medium at the start of fermentation based on the total amount of fermentation medium may be, for example, 0.1% to 40%, 1% to 30%, or 8% to 13%. In addition, the concentration of honey as a solid content in a fermentation medium at the start of fermentation based on the total amount of fermentation medium may be, for example, 0.08% to 32%, 0.08% to 24%, or 6.4% to 10.4%.

The concentration of chamomile flower as a solid content in a fermentation medium at the start of fermentation based on the total amount of fermentation medium may be, for example, 0.01% to 20%, 0.10% to 5%, or 0.5% to 2%. The mass ratio of honey (total amount) to chamomile flower (dry mass) in a fermentation medium at the start of fermentation may be, for example, 4000:1 to 1:200 or 26:1 to 4:1. A fermentation medium may further contain well-known nutrients required for culture of lactic acid bacteria, for example, vitamins, amino acids, minerals, salts, surfactants, fatty acids, and metals. Honey used for fermentation may be sterilized through heating or the like.

Lactobacillus fermented extract of honey or Lactobacillus fermented extract of honey and chamomile flower may be used as is obtained through fermentation or may be subjected to treatments such as heating, filtration, purification, concentration, evaporation to dryness, freeze-drying, and spray-drying. Lactobacillus fermented extract of honey or Lactobacillus fermented extract of honey and chamomile flower may contain live and/or dead bacterial cells of Lactobacillus, and bacterial cells may be separated and removed through filtration or the like.

The content of Lactobacillus fermented extract of honey in the composition according to the present embodiment may be, for example, 25 mass % or more, 26 mass % or more, 27 mass % or more, 28 mass % or more, or 29 mass % or more, and may be, for example, 35 mass % or less, 34 mass % or less, 33 mass % or less, 32 mass % or less, or 31 mass % or less or 30 mass % or less.

The composition according to the present embodiment may be used as, for example, cosmetic or cosmetic raw materials.

Cosmetic raw materials may be in any form such as a solid, a liquid, or a paste. Cosmetics may be medicinal cosmetics (in other words, quasi-drugs). Cosmetics include any products that can be applied to the skin of animals (especially humans).

Cosmetics may contain components acceptable as cosmetics in addition to honey, royal jelly, butylene glycol, pentylene glycol, Lactobacillus fermented extract of honey, or Lactobacillus fermented extract of honey and chamomile flower. Examples of components acceptable as cosmetics include whitening agents, moisturizers, antioxidants, oily components, ultraviolet absorbers, surfactants, thickeners, alcohols, powder components, colorants, aqueous components, water, and various skin nutrients.

The dosage form of cosmetics may be, for example, a solubilized type, an emulsified type, a powder type, an oil liquid type, a gel type, an ointment type, an aerosol type, a water-oil two-layer type, or a water-oil-powder three-layer type. The cosmetics may be, for example, basic skin care products such as facial cleansers, skin lotion, emulsion, cream, gel, essence, serums, pack, mask, mist, and UV preventive cosmetics, makeup cosmetics such as foundation, lipstick, cheek rouge, eye shadow, eyeliner, and mascara, massage agents, cleansing agents, after-shave lotion, pre-shave lotion, shaving creams, body soap, soap, hand cream, and hand soap.

In particular, the composition according to the present embodiment is suitably used as a booster serum (introduction liquid (introduction serum)) or as a raw material thereof. A booster serum is a product that improves skin compatibility of cosmetics such as emulsion and skin lotion applied after applying the serum. The composition according to the present embodiment improves skin compatibility, and therefore can be suitably used in cosmetics for such purposes.

Regarding the dosage of the composition according to the present embodiment for parenteral administration, the composition may be administered once a day or may be administered while being divided into a plurality of times, such as twice a day or three times a day. The dosage may vary depending on the health condition of an application target, the combination with other compositions, or the site and range of application, and therefore can be set arbitrarily by those skilled in the art as necessary. For example, the application amount when administered once to the skin may be 0.001 to 10 mg/cm², preferably 0.001 to 8 mg/cm², and more preferably 0.001 to 6 mg/cm².

A method for producing the composition according to the present embodiment is not particularly limited and can be carried out according to a well-known method as appropriate. For example, a composition used for the above-described purposes can be obtained by mixing honey or, if necessary, royal jelly, butylene glycol, pentylene glycol, or Lactobacillus fermented extract of honey with an intermediate or final product in the process of producing cosmetics.

### [Method for Improving Skin Compatibility]

A method for improving skin compatibility according to one embodiment includes applying the above-described composition comprising honey to the skin. As mentioned above, a contact angle of water on the skin to which the composition comprising honey has been applied is 30 degrees or less. The application of the above-described composition comprising honey improves the skin compatibility better than when the composition comprising honey is not applied to the skin. The method for improving the above-described skin compatibility may include further applying the above-described royal jelly, the above-described butylene glycol, and the above-described pentylene glycol to the skin. In this case, a composition comprising the above-mentioned honey may further contain these components and be applied to the skin. In addition, the method for improving the above-described skin compatibility may include further applying the above-described Lactobacillus fermented extract of honey. In this case, the composition comprising the above-mentioned honey may further contain the above-mentioned Lactobacillus fermented extract of honey and be applied to the skin. The Lactobacillus fermented extract of honey may be obtained through fermentation of honey and chamomile flower with Lactobacillus kunkeei. The honey may be Manuka honey and/or acacia honey.

A method for improving skin compatibility according to another embodiment is a method for improving skin compatibility by applying a composition to the skin, comprising blending honey into a composition, and in which a contact angle of water on the skin to which the composition has been applied is 30 degrees or less. The method may further comprising blending based on the total amount of the composition, 5% by mass or more and 60% by mass or less of honey, 0.001% by mass or more and 0.5% by mass or less of royal jelly, 0.1% by mass or more and 10% by mass or less of butylene glycol, 0.01% by mass or more and 2.5% by mass or less of pentylene glycol, and 25% by mass or more and 35% by mass or less of a Lactobacillus fermented extract of honey into the composition. Other specific aspects of the method may be the same as those of the method for improving skin compatibility according to the embodiment described above.

### EXAMPLES

Hereinafter, the present invention will be described in more detail based on examples. However, the present invention is not limited to the following examples.

### Test Examples: Evaluation of Skin Compatibility Improvement Effect by Measuring Water Contact Angle

The following procedure was followed to measure the contact angle of water on the skin to which each sample shown in Table 1 was applied and to evaluate the effect of improving skin compatibility.

0.1 g of samples 1 to 5 shown in Table 1 were applied onto each Bio Skin Plate (manufactured by Beaulax Co., Ltd.) within 5 cm×5 cm and then dried for 1 hour or longer. Next, 2 μL of water was added dropwise onto the areas where each of the above-described samples was applied, and the water contact angle after 1,000 milliseconds was measured through a liquid droplet method. The measurement of the water contact angle was analyzed through a θ/2 method with an automatic contact angle meter DMo-602 (manufactured by Kyowa Interface Science Co., Ltd.). The results thereof are shown in Table 2.

TABLE 1

| Sample | |
|---|---|
| name | Components and composition (w/w %) |
| Sample 1 | Manuka honey (100%) |
| Sample 2 | Acacia honey (100%) |
| Sample 3 | Royal jelly extract (0.8%), butylene glycol (15%), |
| | pentylene glycol (5%), water (79.2%) |
| Sample 4 | Lactobacillus/(honey/chamomile flower) fermented liquid |
| | (88%), butylene glycol (10%), pentylene glycol (2%) |
| Sample 5 | Mixture of Sample 1, Sample 3, and |
| | Sample 4 at a weight ratio of 1:1:1 |

TABLE 2

| | Non- | Sample | Sample | Sample | Sample | Sample |
|---|---|---|---|---|---|---|
| | application | 1 | 2 | 3 | 4 | 5 |
| Contact | 92 | 29.7 | 28.5 | 14.5 | 23.3 | 12.6 |
| angle (°) | | | | | | |

In the case of non-application, the contact angle of water on the skin was 92 degrees. On the other hand, the contact angle of water on the skin after applying Samples 1 and 2 became 30 degrees or less. This suggests that the application of honey to the skin improves subsequent skin compatibility. In addition, the contact angle of water on the skin after applying Sample 5 was smaller compared to the contact angle of water on the skin after applying Samples 1 and 2. This suggests that the skin compatibility is further improved by applying royal jelly, butylene glycol, pentylene glycol, and Lactobacillus fermented extract of honey and chamomile flower, in addition to honey, to the skin.

What is claimed is:

1. A composition for improving skin compatibility, comprising:
honey,
wherein a contact angle of water on the skin to which the composition has been applied is 30 degrees or less.

2. The composition according to claim 1, further comprising:
royal jelly;
butylene glycol; and
pentylene glycol.

3. The composition according to claim 2, further comprising:
Lactobacillus fermented extract of honey.

4. The composition according to claim 3,
wherein the Lactobacillus fermented extract of honey is obtained through fermentation of honey and chamomile flower with Lactobacillus kunkeei.

5. The composition according to claim 1,
wherein the honey is Manuka honey and/or acacia honey.

6. The composition according to claim 3, which comprises based on the total amount of the composition, 5% by mass or more and 60% by mass or less of honey, 0.001% by mass or more and 0.5% by mass or less of royal jelly, 0.10% by mass or more and 10% by mass or less of butylene glycol, 0.01% by mass or more and 2.5% by mass or less of pentylene glycol, and 25% by mass or more and 35% by mass or less of a Lactobacillus fermented extract of honey.

7. A method for improving skin compatibility, comprising applying the composition according to claim 1 to the skin.

8. The method according to claim 7,
wherein the composition comprising:
royal jelly;
butylene glycol; and
pentylene glycol.

9. The method according to claim 8,
wherein the composition further comprising:
Lactobacillus fermented extract of honey.

10. The method according to claim 9,
wherein the Lactobacillus fermented extract of honey is obtained through fermentation of honey and chamomile flower with Lactobacillus kunkeei.

11. The method according to claim 7,
wherein the honey is Manuka honey and/or acacia honey.

12. The method according to claim 9,
wherein the composition comprises based on the total amount of the composition, 5% by mass or more and 60% by mass or less of honey, 0.001% by mass or more and 0.5% by mass or less of royal jelly, 0.1% by mass or more and 10% by mass or less of butylene glycol, 0.01% by mass or more and 2.5% by mass or less of pentylene glycol, and 25% by mass or more and 35% by mass or less of a Lactobacillus fermented extract of honey.
